(19) United States
(12) Patent Application Publication
(10) Pub. No.: US 2025/0276137 A1
(43) Pub. Date: Sep. 4, 2025

(54) MEDICAL PUMP FOR ENDOSCOPY

(71) Applicant: W.O.M. WORLD OF MEDICINE GMBH, Berlin (DE)

(72) Inventors: Matthias HILDEBRAND, Berlin (DE); Stefan KÜRBIS, Mahlow (DE); Carl CHAMBERS, Berlin (DE); Holger WINTERBERG, Berlin (DE); Colin M. KRÜGER, Berlin (DE)

(21) Appl. No.: 18/561,446

(22) PCT Filed: May 15, 2022
(86) PCT No.: PCT/IB2022/054513

§ 371 Date: Apr. 17, 2024

(30) Foreign Application Priority Data
May 17, 2021 (DE) 10 2021 002 547.4

(51) Int. Cl. A61M 13/00; A61M 1/00

(52) U.S. Cl. CPC A61M 13/00; A61M 1/80; A61M 2205/3331

(57) ABSTRACT

The present invention relates to a medical pump for endoscopy with a pressure controller, wherein the pressure controller can detect muscle contractions of the person being treated and can emit a corresponding warning signal.

### BACKGROUND OF THE INVENTION

The present invention relates to a medical pump for endoscopy with a pressure controller, wherein the pressure controller can detect muscle contractions of the person being treated and can emit a corresponding warning signal.

It is well known that during endoscopic examinations and especially during therapeutic interventions, the respective body cavity is expanded by fluid inflow. During laparoscopy, a gas (preferably CO₂) is usually introduced into the abdomen, creating an internal pressure that is higher than the external pressure. In this way, the abdomen is stretched to create space for the introduction of the surgical equipment. Modern systems also have a suction device in order to be able to quickly remove visually impairing smoke gases, but also to keep the pressure in the body cavity as constant as possible during the operation. These systems are therefore designed to keep the pressure in the body cavity as constant as possible.

During operations, especially lengthy operations, the patient may become less sedated and respond to tissue irritation with muscle contractions. This results in an increase in internal pressure of up to 60 mm Hg, causing the medical pressure maintenance system to increase the suction rate to reduce the pressure. This quickly returns the internal pressure to the target value. However, once the muscle contractions subside, the pressure continues to drop abruptly, which may cause the body cavity to collapse. The medical staff treating the patient must then wait for the pressure to build up again. This not only delays the course of the operation, but can also be problematic if, e.g., vessels have been damaged during the last stage of the operation, causing blood to leak out before the vessels can be closed again.

To overcome this technical problem, the medical device described below is proposed, which detects the increase in pressure caused by muscle contraction and emits a warning signal.

### SUMMARY OF THE INVENTION

The medical pump for endoscopy according to the invention is capable of detecting pressure peaks caused by muscle contraction. For this purpose, first the pressure in the body cavity is monitored, as already described in the prior art. The pressure data is stored in the device. In addition, the operating parameters of the supply pump and the suction pump (in particular the gas flow) are monitored and also stored. In addition, the operating parameters of the endoscopic instruments, such as endoscopes, catheters and in particular RF instruments, e.g., coagulation electrodes, are monitored. If the system detects an increase in pressure, the operating parameters of the above devices are checked: For example, if the system detects an increase in pressure after the supply pump has increased the inflowing gas flow without the exhaust pump also increasing its gas flow, the system will perform normal pressure regulation, for example, increasing the exhaust flow. The same applies if the exhaust capacity is reduced while the supply flow remains constant.

For example, an increase in pressure could be caused by an occlusion of the suction trocar. However, this could be detected by a decrease in gas flow in the suction line.

On the other hand, if the system detects an increase in pressure without a change in the gas flow parameters, it can be assumed that the increase in pressure was caused by muscle contractions. In this case, a warning signal is triggered. The warning signal may be visual or audible, for example, a message on the display such as “check relaxation”. The clinical staff can then adjust the sedation if necessary. The clinical staff will of course check to see if there is an external reason for an increase in pressure, such as manual pressure on the abdomen.

First of all, an insufflator according to the invention includes a standard connection to a pressurized gas cylinder containing a gas suitable for medical purposes, e.g., carbon dioxide. The gas is delivered to a trocar via a proportional valve and a sterile tube. Inside the insufflator, both pressure and gas flow are measured in the line. An optional filter to retain particles is also included.

A second trocar is connected to a suction pump (desufflation pump) integrated into the insufflator. Optionally, a filter is connected in between that can absorb particles, droplets, and/or toxic gases. The pumped desufflation gas can then be released into the atmosphere. The suction pump can also be controlled and includes a measurement of the gas flow in the suction line. The insufflator is controlled by a switching unit that can control both the flow of gas supplied and the flow of gas removed.

### BRIEF DESCRIPTION OF THE FIGURES

FIG. 1 shows an embodiment of an insufflator according to the invention.

FIG. 2 shows a device according to the invention in which the suction pump (12) runs continuously and the suction power is controlled by a bypass valve (14).

FIG. 3 shows another variant of the device according to the invention. Here, instead of an internal suction pump, a connection (16) for an external pump is provided.

### DETAILED DESCRIPTION OF THE INVENTION

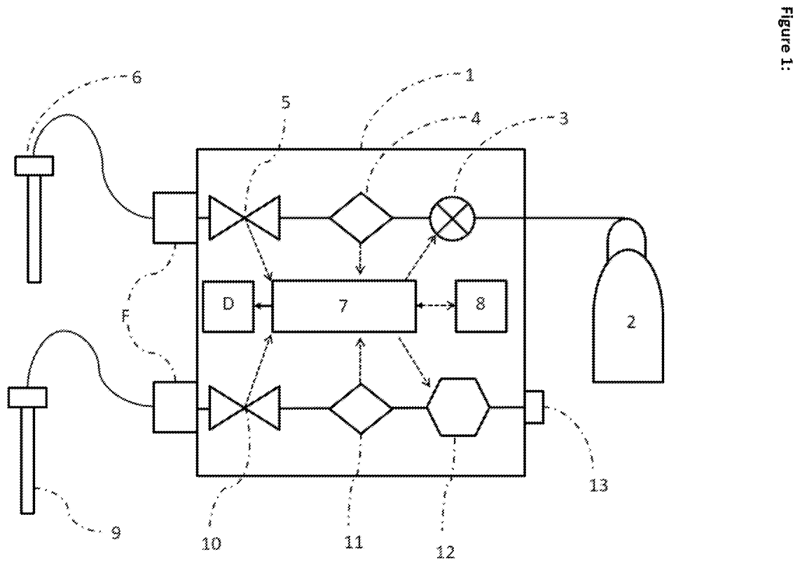

As shown in FIG. 1, the insufflator is connected to the patient using two tubes. The first tube is used for insufflation. Gas is supplied to the patient during operation to build up the pressure in the abdomen. This tube is also used to measure abdominal pressure. The second tube is used to connect the suction pump to the patient for smoke evacuation, for example.

Electronically controlled pumps, such as those described in the device according to DE 102013016063 or similar publications, can be used as suction pumps.

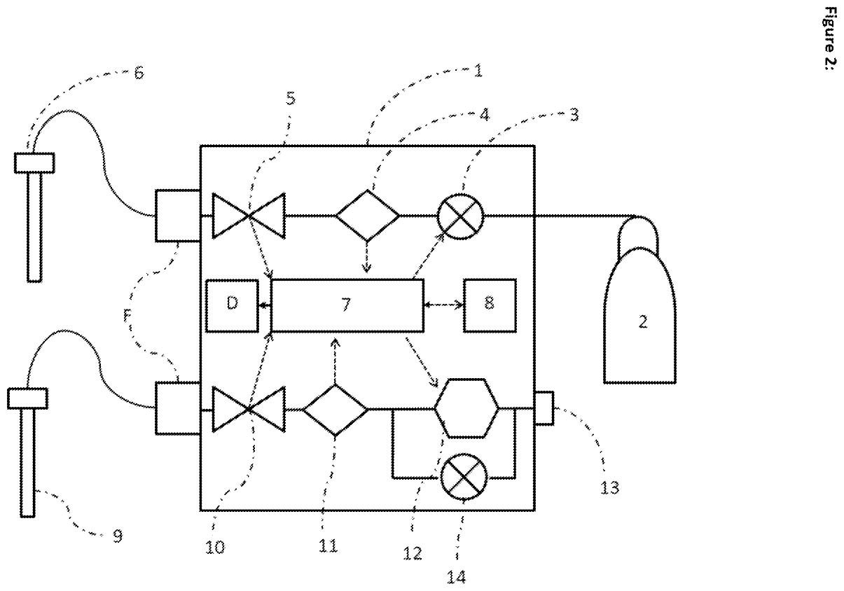

Alternatively, for example, the suction pump can be controlled via a bypass valve (FIG. 2). For example, the pump can be set to a specific output that is largely constant, and the output is then controlled via the bypass valve.

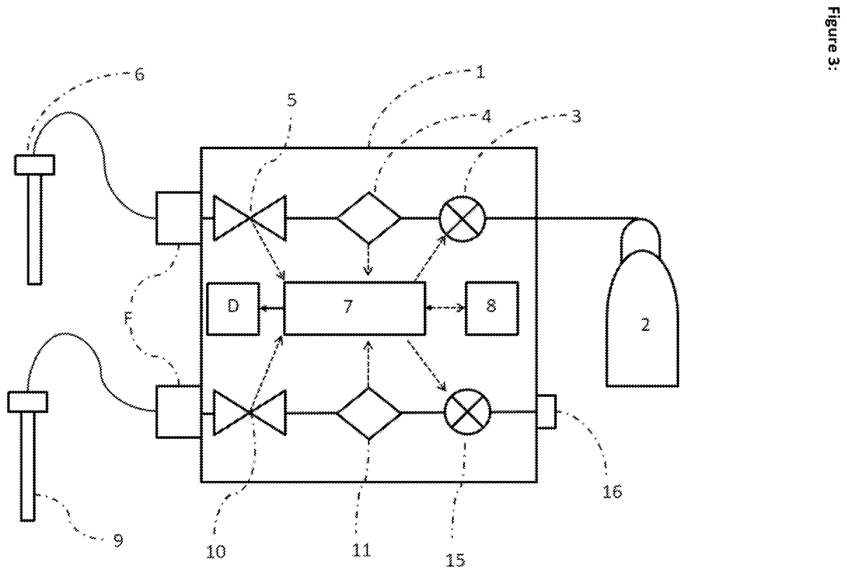

Alternatively, a control valve can also be placed directly in the suction line (FIG. 3). In this way, even external pumps can be used, for example, the wall suction system available in the operating room. The control unit of the insufflator then regulates the suction power via the control valve shown.

In addition, the insufflator may contain sensors (optional) that can monitor the operation of endoscopic instruments, e.g., electrocoagulation devices. All recorded measurement data is stored and fed to a computer unit, which performs the necessary data analysis using specially designed software. The alarm is triggered when the pressure rises above a defined threshold value above the respective target value, which is not due to changes in the pump parameters. For devices according to the invention, a corresponding threshold value of 20 mm Hg above the target value has proven to be excellent. Deviating settings of the threshold value to, for example, 10, 30, 40, or 50 mm Hg are possible.

It is also possible to record the duration of the increase in pressure. Often, short contractions (less than 10 seconds) occur first, resulting in a correspondingly short increase in pressures (pressure pulses) above the preset threshold value. The detection of at least two pressure pulses should then trigger the alarm.

In the event of a prolonged increase in pressure caused by muscle contraction (more than 15-20 seconds), the pressure controller of the device can not only trigger an alarm, but also reduce the pressure until the cramp is relieved. For this purpose, it is recommended not to reduce the pressure completely to the target value, but to maintain a slight overpressure. When the threshold value of 20 mm Hg above the target value is reached, the pressure can be temporarily adjusted to 10 mm Hg above the target value. This temporary overpressure accelerates the build-up of pressure, especially in larger body cavities (such as the abdomen).

Optionally, it is also possible to include the dynamics of the increase in pressure to determine a muscle contraction. The muscle contractions induced here lead to a relatively fast increase in pressure compared to the regular increase in pressure caused by a change in the insufflation or desufflation parameters. Therefore, evaluating the rate of increase in pressure allows a more accurate classification of the causes of the increase in pressure.

FIG. 1 shows an embodiment of an insufflator according to the invention. The insufflator (1) is connected to a gas source (2), e.g., in the form of a CO₂ gas cylinder. The gas is supplied to the insufflation trocar (6) via a proportional valve (3), a pressure sensor (4), a volume flow sensor (5), and a filter (F). The desufflation trocar (9) is connected to the insufflator via a tube, where the gas flow first passes through a filter (F), a volume flow sensor (10), and a pressure sensor (11) to a suction pump (12). The output of the suction pump is connected to a device outlet (13). The device outlet (13) can of course be equipped with an additional filter. The measured data from the pressure sensors (4, 11) and the volume flow sensors (5, 10) are transmitted to the computer unit (7) with a connected memory (8). The computer unit (7) controls the proportional valve (3) and the suction pump (12). As the person skilled in the art will recognize, the positions of the pressure sensors and the volumetric flow sensors may also be different: of course, it is possible for the insufflation flow to pass first through the volumetric flow sensor (5) and then through the pressure sensor (4). Similarly, the volumetric flow sensor (10) of the desufflation line may be located behind the suction pump (12) in the direction of flow. In any case, a signal is output to the display (D) when an increase in pressure of more than 20 mm Hg due to muscle contraction is detected. As an alternative or in addition, the signal may also be audible.

FIG. 2 shows a device according to the invention in which the suction pump (12) runs continuously and the suction power is controlled by a bypass valve (14). The bypass valve (14) is also controlled by the computer unit (7) (not shown in FIG. 2).

FIG. 3 shows another variant of the device according to the invention. Here, instead of an internal suction pump, a connection (16) for an external pump is provided. Many hospitals are equipped with such pumps that can be used for the intended application of an insufflator according to the invention. In this case, only a control valve (15) is required to regulate the suction rate of the external pump (not shown).

The individual components of the device according to the invention are for the most part already known from earlier publications such as U.S. Pat. Nos. 6,299,592, 5,411,474, WO 1996001132A1, WO 2011041387A1, U.S. Pat. No. 5,800,381, DE 4219859B4, DE 10 2013 016 063 A1. Relevant methods of operation are also disclosed in these publications. A suitably programmed microcomputer with associated memory and input and output devices serves as the control unit. Volume flow sensors are already known from other medical devices (e.g., in the context of ventilation devices), so that they need not be discussed further here.

The person skilled in the art may implement alternative and/or additional embodiments of the invention without being inventive.

1. An insufflation device for use in medical technology comprising
an insufflator for introducing insufflation gas into a body cavity,
a control unit,
an insufflation line and a desufflation line,
wherein the desufflation line is connected to a suction pump,
wherein the insufflation line and the desufflation line each have a pressure sensor and a volume flow sensor,
wherein the insufflator displays a warning signal when the pressure in the body cavity exceeds a threshold value above the set target value without a change in the volume flows being able to cause the increase in pressure.

2. The insufflation device for use in medical technology according to claim 1, wherein the threshold value is 10, 20, 30, 40, or 50 mm Hg.

3. The insufflation device for use in medical technology according to claim 1, wherein the insufflator dis-plays an warning signal when the pressure in the body cavity several times briefly exceeds a threshold value above the set target value without a change in the volume flows being able to cause the increase in pressure.

4. The insufflation device for use in medical technology according to claim 1, wherein the insufflator evaluates the dynamics of the increase in pressure as it measures the pressure in the body cavity.
